(19) United States
(12) Patent Application Publication
(10) Pub. No.: US 2026/0090829 A1
(43) Pub. Date: Apr. 2, 2026

(54) INSTRUMENTS DE CHIRURGIE ORTHOPÉDIQUE VÉTÉRINAIRE D'INSTALLATION D'IMPLANTS OSSEUX EXPANSIBLES

(71) Applicant: IN LIFE VET SA, Bulle (CH)

(72) Inventor: Guillaume LACAZE, La Rippe (CH)

(73) Assignee: IN LIFE VET SA, Bulle (CH)

(21) Appl. No.: 19/340,562

(22) Filed: Sep. 25, 2025

(30) Foreign Application Priority Data
Sep. 25, 2024 (EP) 24202393.5

(51) Int. Cl. A61B 17/70; A61B 17/00; A61B 17/88

(52) U.S. Cl. CPC A61B 17/7071; A61B 17/7065; A61B 17/8833; A61B 2017/00898

(57) ABSTRACT

An instrument for implanting expandable bone implants, by expanding between folded configuration and deployed configurations of the implant extending along a longitudinal axis between a proximal end adapted to engage with the implantation instrument for holding the implant and a distal end intended to be firstly inserted into the bone, the instrument including a main part grippable by a practitioner, an implant-holding portion including a gripping tube extending along the longitudinal axis and having, at its distal end, retention means that complement engagement means for engaging the implant, for holding the implant by its proximal end, the instrument further comprising an injection instrument for injecting fluid into said implant, having a cavity for receiving the fluid, the injection instrument being behind the implant-holding portion along the longitudinal axis and having a cannula for conveying the fluid to the inside of the implant while still held by the implant-holding portion.

The present application relates to the field of surgery, in particular veterinary orthopaedic surgery and notably to the treatment of a collapsed bone structure by restoring the volume of (or correcting) this bone structure. The present application particularly relates to instruments for installing expandable bone implants for repairing or restoring damaged bone structures, notably in the spine for the treatment (often called “reduction”) of compression fractures, notably vertebral compression fractures (VCF).

In this field, the problem of restoring the volume of bone structure that has collapsed is well known and the literature contains an abundance of solutions using expandable implants capable of transitioning from a folded configuration to a deployed configuration in order to restore the height of the bone structure, preferably in combination with the injection of bone substitute cement, called cement (or bone cement). Many cements are known and they all have the advantage of being injectable in a liquid or viscous state for a certain period of time, then of hardening (by polymerisation) inside the bone structure in order to stabilise it.

Significant problems in this field relate to the expansion of the implant in order to restore the height of the damaged bone tissue as well as to cement leakage, but also to the installation of implants in bones, notably vertebrae. Indeed, instruments need to be available that facilitate implantation and reduce the duration and the difficulty of the task for practitioners as much as possible.

Numerous solutions are known from the prior art, notably such as patent applications EP 3086729, US 11540926, EP 3747385, EP 2572680, EP 3958752, EP 2693967, EP 2405835, US 9579130, EP 4216836, WO 2023/122005, WO 2022/162418, EP 3843668 or U.S. Pat. No. 10,945,861, but these solutions exhibit various problems relating to difficulties in handling for deployment, and of stability and reliability once deployed. Moreover, these known solutions are generally accompanied by an injection of bone cement, but do not provide any teaching relating to cement leakage outside the implant, whereas such leakage can be detrimental to the surrounding tissues, or even to the entire organism if the chemical substances in the cement enter the bloodstream. Indeed, the cement generally comprises one or more polymerisable chemical substances, for example, such as poly(methyl methacrylate) (PMMA) and possibly additives. In addition, the temperature reached during the polymerisation of the cement is not harmless since it is generally greater than 60°.

Devices for correction and stabilisation (or bone fracture reduction), notably of the spine in the form of a stent, or in the form of porous inflatable bags or of balloons, as in documents EP 1408888 or EP 1379185, possibly equipped with support flanges, as in document US 2006/0100706, are known from the prior art, notably from documents EP 1308134, U.S. Pat. No. 9,510,877, US 8936627 or EP 2467099. Numerous documents propose this type of stent, i.e., a deformable endoprosthesis similar to vascular stents, extenders or endoprostheses, which are generally in the form of a meshed tubular body, which is usually metallic and can be deformed by introducing an inflatable balloon in order to expand the body by spreading apart the meshes, with the balloon then being removed to allow cement to be injected, which hardens and thus forms a correcting and stabilising structure. However, these devices have the disadvantage of requiring implantation in two or even three stages, with the inflation of the balloon and then the injection of cement, which slows down and com-plicates the operation, but also presents a risk of the device collapsing between the deflation of the balloon and when the stent is filled with the cement, in addition to requiring several different instruments to be used successively, which involves an increased health risk. Furthermore, these solutions have the disadvantage of not addressing the significant problem of cement leakage.

Solutions using implants with a mesh structure, made of shape-memory metal, which is constrained into a folded shape for insertion into the bone tissue and is capable of expanding spontaneously, when the stress is released and/or under the effect of heat, are also known, notably from documents EP 1938765, EP 2351539 or WO 2004/34924. These solutions have the disadvantage of using expensive alloys and of involving complex manufacturing in order to obtain an adequate shape memory suitable for the intended implantation, which involves additional cost when multiplied by the number of different implants required to cover the various pathological cases, notably by the amplitude of the deformation the shape memory material can undergo. Moreover, the force exerted by the metal returning to its unstressed form is often not sufficient to properly correct the bone structure that has collapsed, or is at least a limiting factor. Furthermore, these solutions also have the disadvantage of not addressing the significant problem of cement leakage.

Solutions using expandable implants that can be expanded using a lever mechanism, like a car jack, to restore the bone structure to a determined height are also known from the prior art, notably from documents EP 2405835, U.S. Pat. No. 9,579,130, EP 2572680 or EP 1956990. These solutions have the advantage of avoiding a risk of collapsing, unlike a stent that is deployed by a balloon that is removed before the cement is injected, but also have the disadvantage of implantation in two or even three stages and of the fact that the dimensions of the support surface for exerting the expansion force on the bone tissue are limited, notably compared with stents. Furthermore, they also have the disadvantage of being expensive, difficult to deploy, of not being adjustable to the morphology of bone structures, and of also not addressing the significant problem of cement leakage.

Other recurring problems in orthopaedic surgery relate to invasiveness (i.e., the aim of making the smallest possible incision and of minimising lesions), but also to the deployment ratio in order to obtain a deployed implant that fills the largest possible volume, while having been introduced through the smallest possible passage. Furthermore, this deployment ratio will impact the distribution of forces for correcting the vertebrae: if the deform-ability is too great, the cement-injection pressure will deform the pouch rather than restore the height.

An additional problem to that of deployment relates to folding, which generally is not possible in the implants of the prior art. Controlling folding allows the deployment to be controlled and therefore allows the injection site to be controlled with a uniform distribution of the cement and of the pressure to fill the space to be filled following the collapse. Perfect proportionality adapts to the fracture, while following the shape of the bone inside the fracture.

Within this context, it is understood that many technical problems remain in the field that are associated with the implants, and which are accompanied by problems relating to the implantation instruments, which are generally too numerous and complex to address the significant issues of invasiveness and the ease and the duration of the surgical intervention.

Within this context, an aim of the present invention is to overcome at least some of the dis-advantages of the prior art by proposing a surgical implantation instrument for expandable bone implants, for restoring a collapsed bone structure, that is reliable and simple to handle and to implant.

This aim is achieved by an instrument for implanting expandable bone implants for veterinary orthopaedic surgery, for restoring the volume and/or the geometry of a bone, by expanding between a folded configuration and a deployed configuration of the implant extending along a longitudinal axis between a proximal end adapted to engage with said im-plantation instrument for holding the implant and a distal end intended to be firstly inserted into the bone, said instrument comprising a main part that can be gripped by a practitioner, an implant-holding portion comprising a gripping tube extending along the longitudinal axis and having, at its distal end, retention means that complement engagement means for engaging the implant, for holding the implant by its proximal end, said instrument being characterised in that it comprises an instrument for injecting fluid, such as bone cement, into said implant, comprising at least one cavity adapted to receive the fluid, said injection instrument being disposed behind the implant-holding portion along the longitudinal axis and comprising at least one cannula for conveying the fluid to the inside of the implant while it is still held by said implant-holding portion.

According to another feature, the instrument also comprises an instrument for expanding the implant adapted to engage with mechanical means for expanding the implant, by virtue of an expansion rod passing through said implant-holding portion and said gripping tube in order to actuate said mechanical expansion means.

According to another feature, said expansion rod passes through said injection instrument to the distal end of the gripping tube.

According to another feature, the retention means for retaining the instrument and their complementary engagement means for engaging the implant comprise a proximal sleeve of the implant, around which said retention means are attached and which comprise a stud that can be moved in an L-shaped groove in order to retain the implant by translational and rotational movement of said stud in said groove relative to the longitudinal axis.

According to another feature, said injection instrument comprises a piston that can be activated on the instrument in order to push the fluid stored in a chamber inside the instrument through said cannula.

According to another feature said said injection cannula is formed by the gripping tube emerging onto a proximal sleeve of the implant.

According to another feature, said injection cannula is formed by said expansion rod, which is hollow and is adapted to be inserted inside the gripping tube and emerges into an opening in a proximal sleeve of the implant retained by the gripping tube, directly into a cavity of the implant or onto a shaft of the implant comprising a conduit and at least one opening for distributing said fluid within the implant.

According to another feature, said injection cannula is formed by said expansion rod, which is hollow and capable of being inserted inside the gripping tube and opening into an opening in a proximal sleeve of the implant held by the gripping tube, directly into a cavity of the implant or onto an axis of the implant comprising a conduit and at least one opening for distributing said fluid into the implant.

According to another feature, the implant-holding portion comprises a mounting secured to the gripping tube directly in the main instrument.

According to another feature, the implant-holding portion comprises a detachable tip having a housing adapted to be mounted on a protrusion of the instrument through which the fluid is conveyed to the detachable tip supporting the gripping tube.

According to another feature, said detachable tip forming the implant holder is retained on the instrument by virtue of a stud engaging with an L-shaped groove for locking in a translational and rotational movement relative to the longitudinal axis.

According to another feature, direction of rotation for locking said implant holder on the instrument is opposite the direction of rotation for locking the implant on the implant holder.

According to another feature, the implant holder comprises a translation latch detachably disposed in said groove in order to prevent any translation movement of said stud, until fluid is injected and/or actuation of the expansion causes said implant to shorten in length.

According to another feature, the implant holder comprises a rotation latch detachably disposed in said groove in order to prevent any rotation movement of said stud, until the uncoupling of the implant holder and the instrument is required.

Another purpose of the present application is to overcome at least some of the disadvantages of the prior art by proposing a surgical intervention system that is easy to use and allows effective stabilization of bone tissue.

This purpose is achieved by an orthopedic treatment system for damaged bone tissue comprising a bone substitute cement and at least one instrument for implanting and injecting cement into the implant, characterized in that it comprises an implant according to one of the embodiments described in this application.

According to another feature, the instrument for implanting and injecting cement comprises means for controlling the pressure and/or suction of the cement in order to fold the implant into a folded configuration if necessary.

Further features and advantages of the present invention will become more clearly apparent upon reading the following description of various embodiments, with reference to the ap-pended drawings, in which:

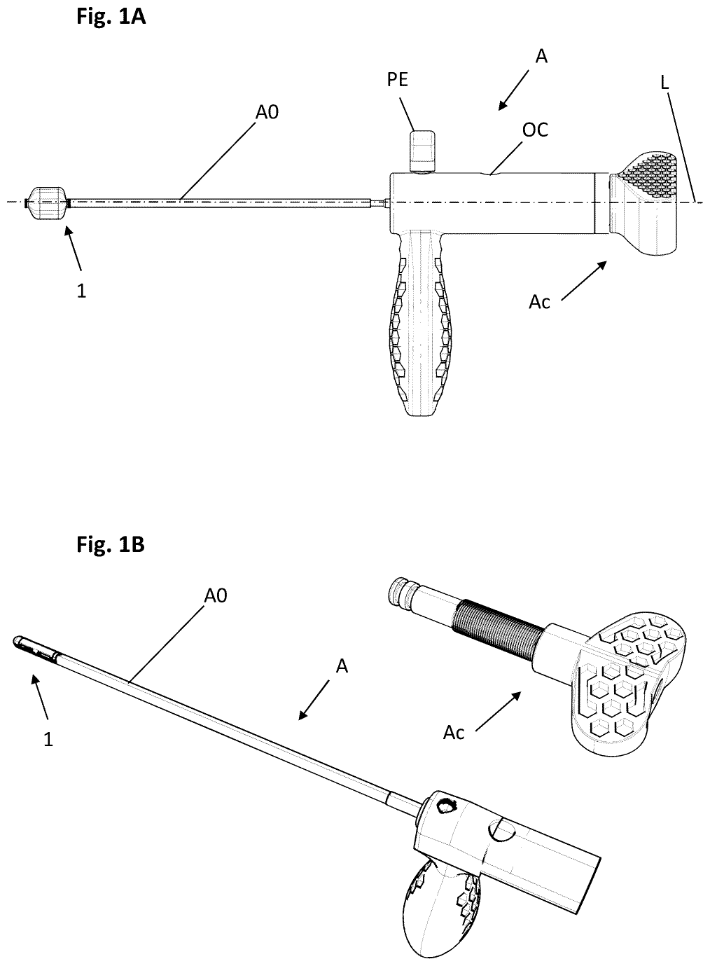

FIG. 1A shows a profile view of instrumentation for implanting an expandable orthopaedic implant according to some embodiments, comprising an implant holder, a cement injection instrument and the implant supported at the end of the implant holder, and FIG. 1B shows a perspective view of the instrumentation of FIG. 1A with the cement injection instrument removed from the implant holder;

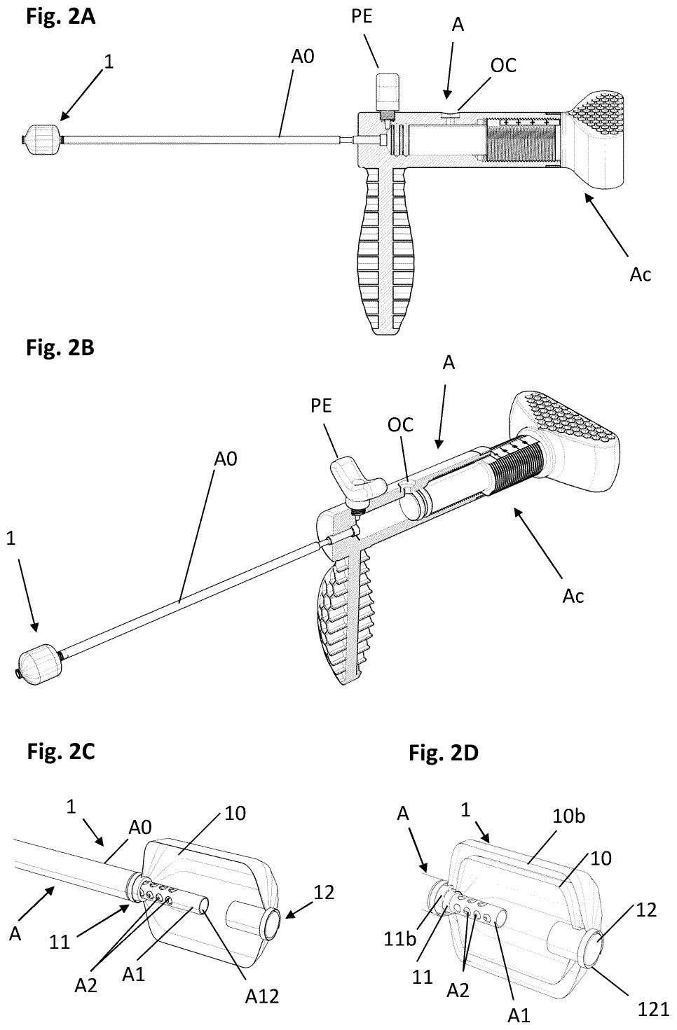

FIG. 2A shows a transparent view of instrumentation for implanting an expandable implant, FIG. 2B shows a perspective view with a partial cross-section of the instrumentation of FIG. 2A, and FIG. 2C shows a perspective view of the end of the implant holder with an implant mounted thereon, with a partial cutaway view of the implant showing the implantation instrument inside the implant according to some embodiments and FIG. 2D shows the same view as FIG. 2B but for a double-layer implant.

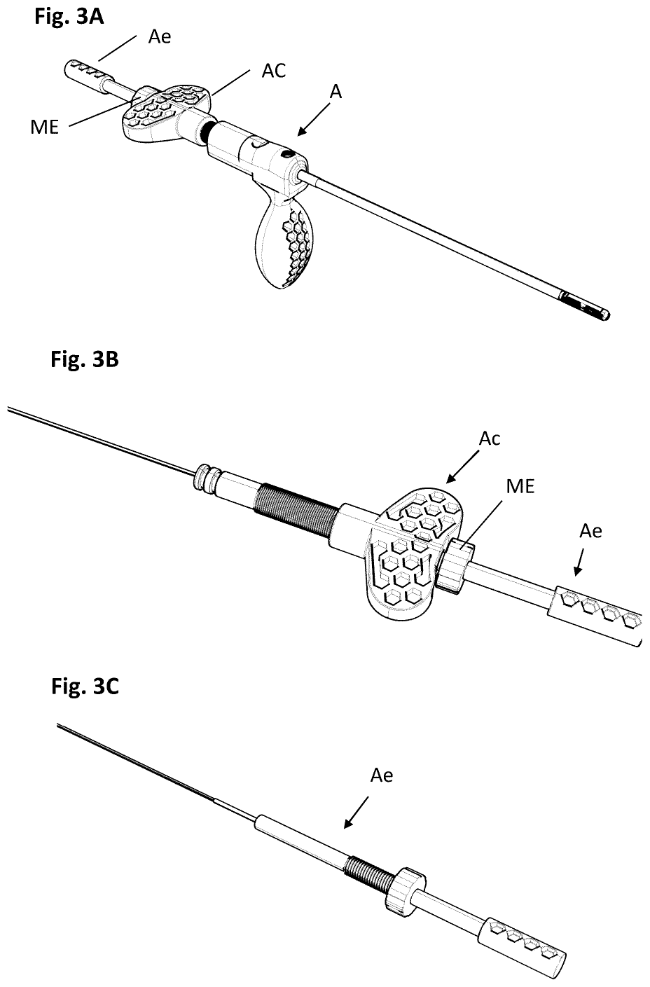

FIG. 3A shows a perspective view of instrumentation for expanding an expandable implant according to some embodiments inserted inside a cement injection instrument, according to some embodiments, FIG. 3B shows the expansion instrument inserted through the injection instrument with the expansion wheel, and FIG. 3C shows the expansion instrument alone;

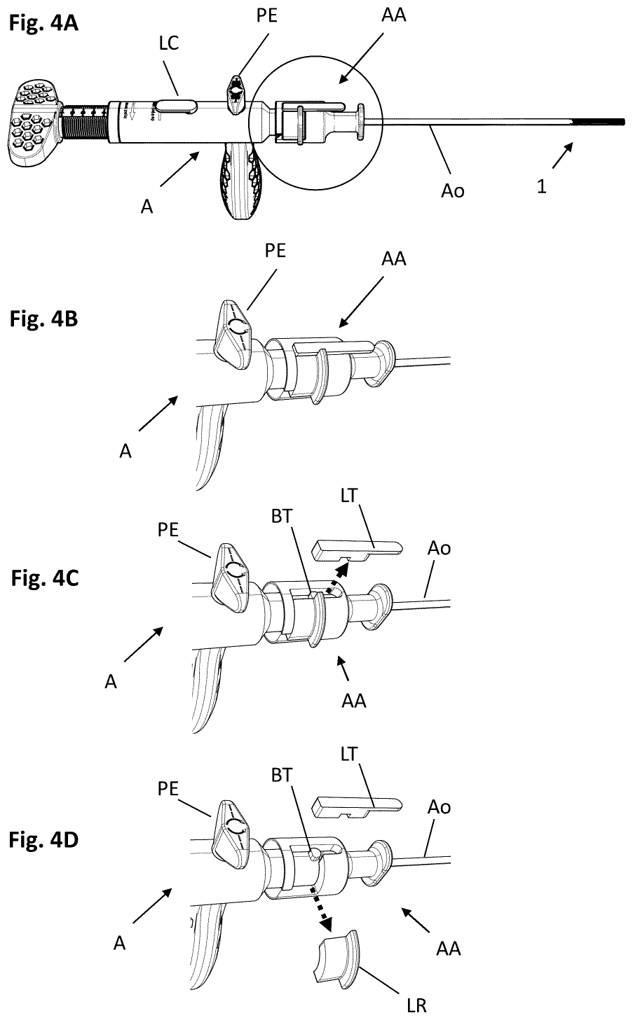

FIG. 4A shows a perspective view of instrumentation for implanting an expandable implant comprising an implant holder mounted on a gripping instrument according to some embodiments, and a cement injection instrument passing through the assembly, FIG. 4B shows details of the circular part indicated in FIG. 4A, and FIGS. 4C and 4D show the same details after the removal of the latches on the implant holder;

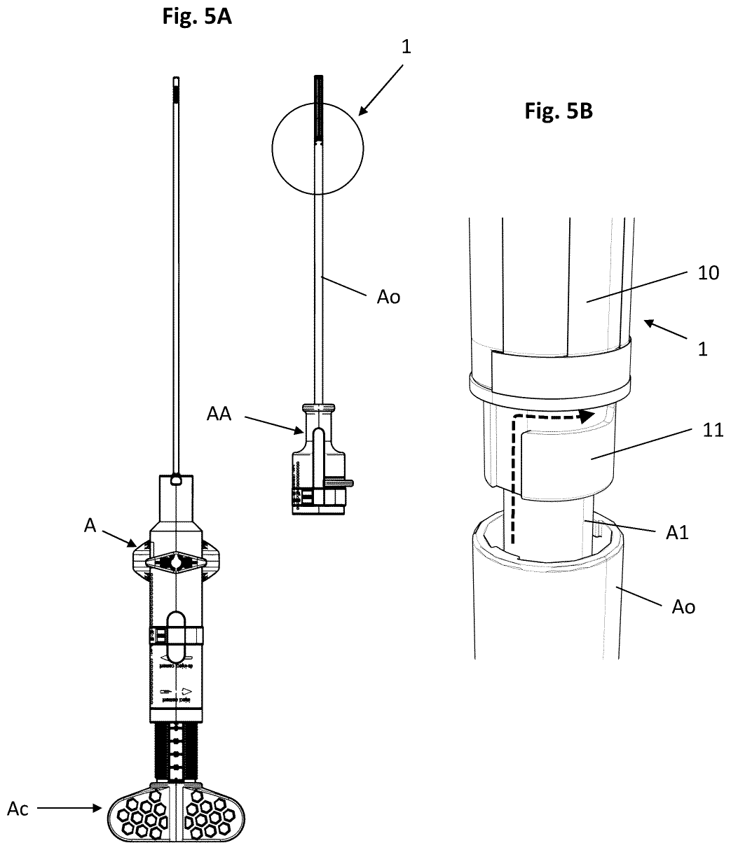

FIG. 5A shows, on the left-hand side, a top view of instrumentation for implanting a bone anchor implant and, on the right-hand side, the implant holder uncoupled from the gripping instrument, and FIG. 5B shows details of the circular zone on the right-hand side of FIG. 5A, with the engagement between the implant holder and the implant, according to some embodiments;

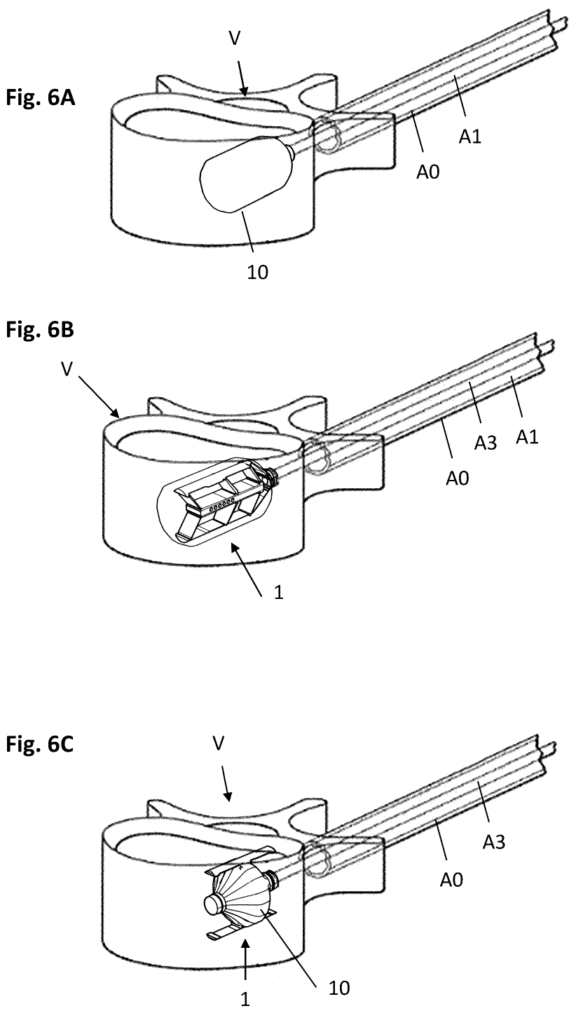

FIGS. 6A, 6B and 6C show perspective views of vertebrae, into which various embodiments of expandable bone implants are inserted;

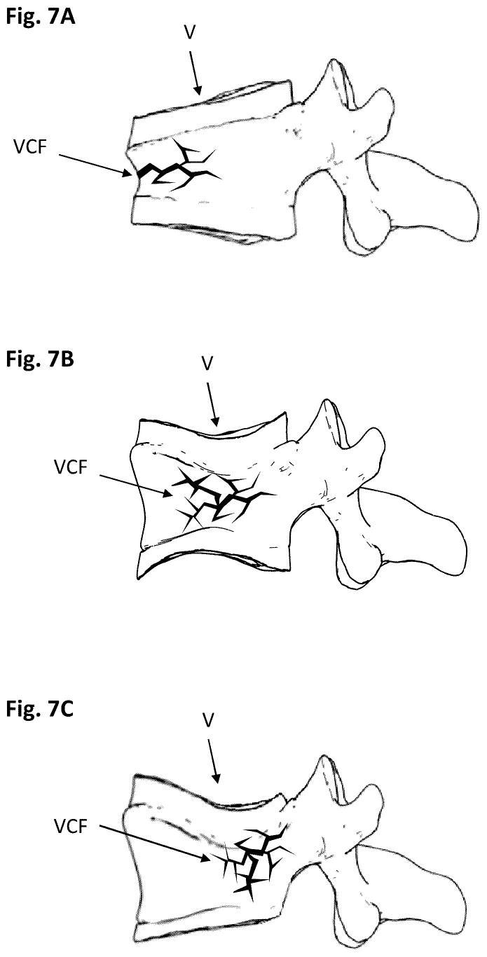

FIGS. 7A, 7B and 7C show profile views of vertebrae that have respectively suffered anterior, median and posterior vertebral compression fractures (VCF);

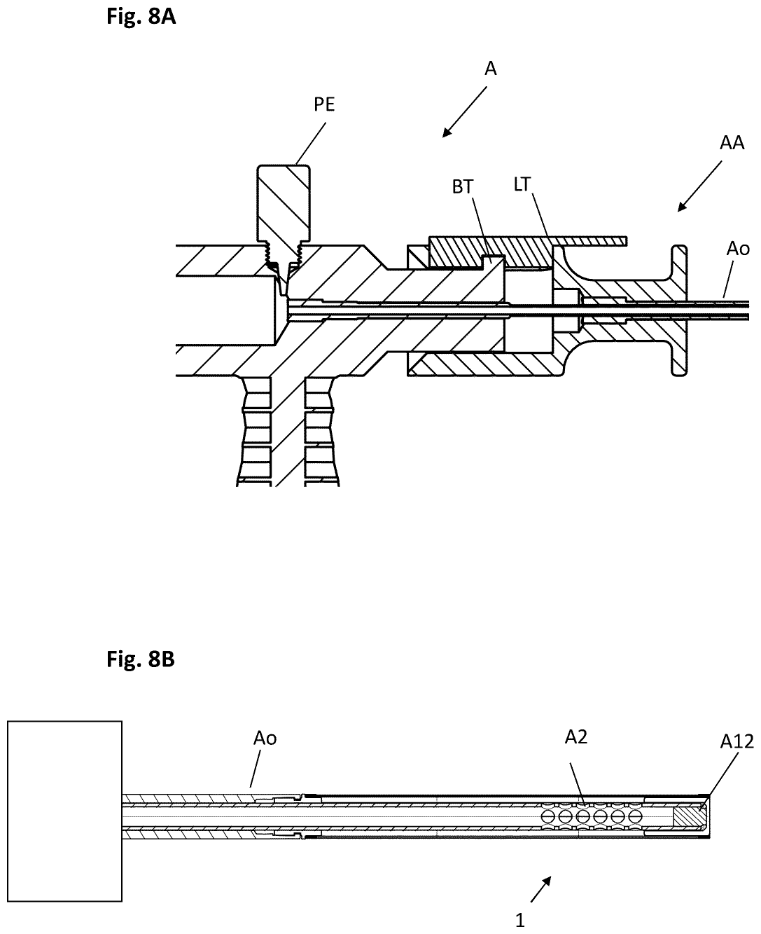

FIG. 8A shows a cross-sectional view of part of instrumentation for implanting an expandable bone implant of the type shown in FIG. 4A, and FIG. 8B shows a cross-sectional view of the implant supported by an instrument of FIG. 8A, according to some embodiments;

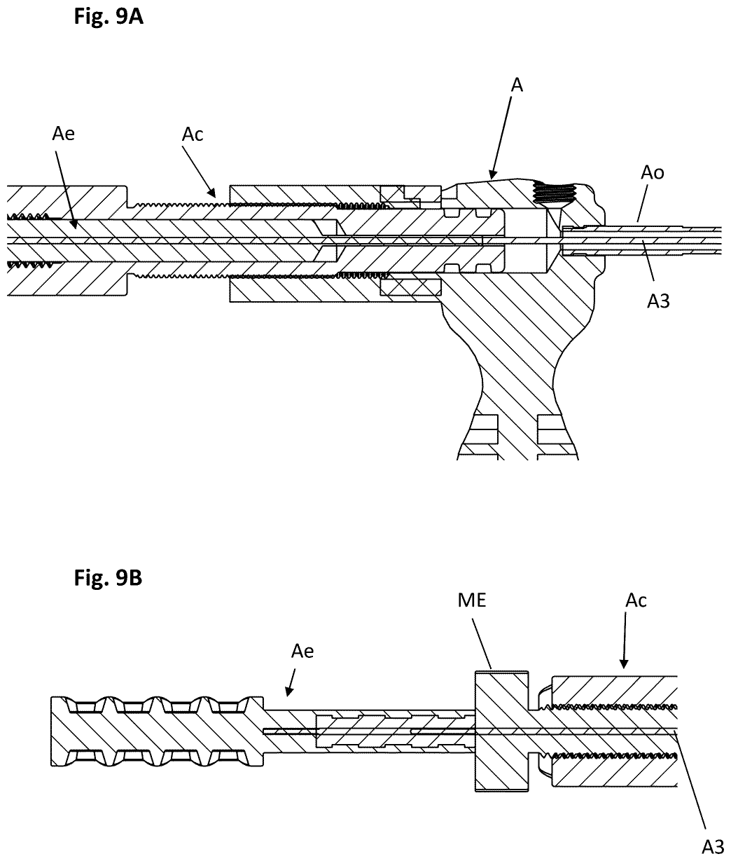

FIG. 9A shows a cross-sectional view of part of instrumentation for implanting an expandable implant of the type shown in FIG. 3A, and FIG. 9B shows a cross-sectional view of the implant held by such an instrument of FIG. 9A, according to some embodiments.

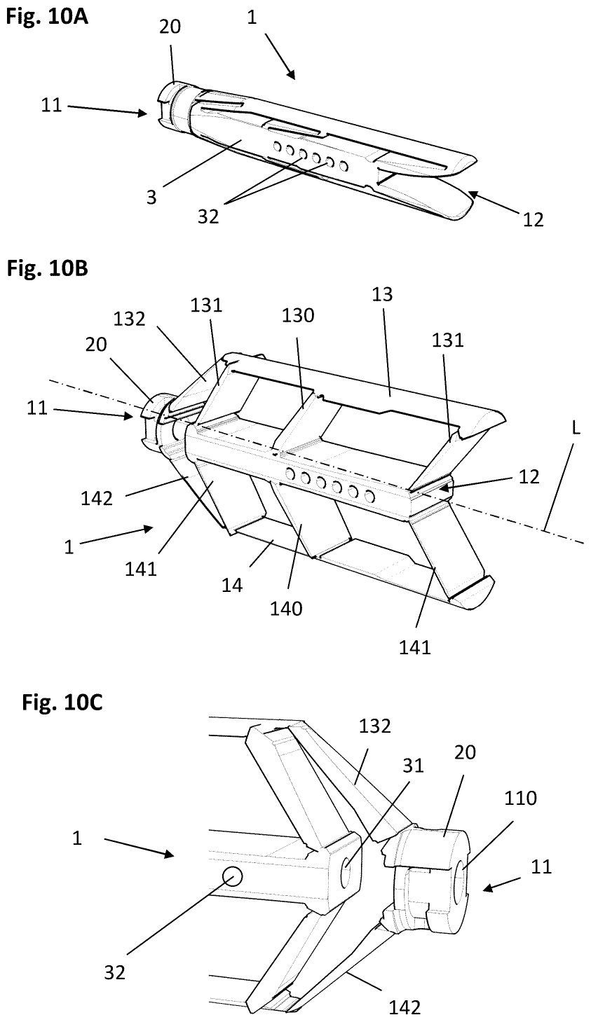

FIG. 10A shows a perspective view of an expandable implant according to certain embodiments in a folded configuration, FIG. 10B shows a perspective view of the implant of FIG. 10A in an expanded configuration, and FIG. 10C shows a detail of the proximal end of the same implant;

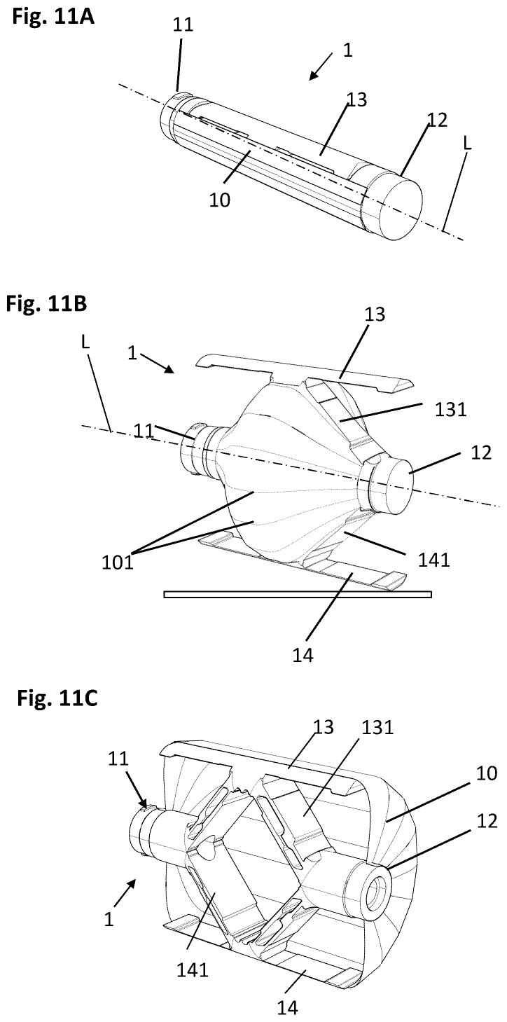

FIG. 11A shows a perspective view of an expandable implant in its folded configuration with an integrated sealed envelope, according to certain embodiments. FIG. 11B shows a perspective view of the same implant in its deployed configuration, and FIG. 11C shows a perspective view of another type of implant whose envelope is not integrated but separate, according to other embodiments, with the implant shown in its deployed configuration with half of its envelope removed to reveal the implant inside.

The present application primarily concerns instrumentation for the implantation of at least an implant and an orthopaedic surgery system for treating a bone fracture and bone tissues in general, and to a method for manufacturing the implant. The bone implant is preferably a spinal implant, and in particular a vertebral or even in fact an intervertebral implant, although other uses can be contemplated elsewhere in the spine (intervertebral discs) or in other bone structures where a space left vacant as the result of a fracture needs to be filled (which can be the result of multiple causes, even though they generally involve a reduction in bone density). Thus, vertebral compression fractures (VCFs) are a preferred application but are not the only conditions that can be treated using the present invention, and a person skilled in the art will appreciate the possibilities that are offered without requiring further details herein. In terms of other bones, the femur or the humerus (head of) can be cited, for example, in the event of a risk of collapse. This application relates to an implant and a veterinary orthopedic surgery system for treating fractured bones and bone tissue in general, as well as a method for manufacturing the implant. The bone implant is preferably a spinal implant, and in particular a vertebral or even intravertebral implant, but other uses are possible elsewhere in the spine (intervertebral spines) or in other bone structures where it is necessary to fill a space left by a fracture (the causes of which may vary, although they generally involve a decrease in bone density). Thus, vertebral compression fractures (VCFs) are a favorite application, but they are not the only ones that can be treated with the present invention, and those skilled in the art will appreciate the possibilities offered without further detail here. Other bones include the femur or humerus (head), for example, in cases where there is a risk of collapse. In the veterinary field, it is known that animals sometimes have bone densities that are very different from those of humans and, above all, vary greatly depending on the species and even on the breed or animals within the same species, particularly in the case of dogs, whose physical properties vary enormously from one breed to another. For example, dachshunds and similar breeds have long (tall) vertebrae but are not very wide compared to other species. It is therefore useful to have expandable implants that allow for significant expansion in height while having a reduced length, and it is clearly necessary to take into account the differences in the shapes and sizes of the bones of different species in order to adapt the therapy effectively with suitable implants. In addition, some species, such as cats, have very rigid cortical bone but more flexible cancellous tissue than other species. It is therefore also necessary to take into account the nature of the bone tissue. Finally, another notable example concerns horses, which have bones, particularly vertebrae, with a specific anatomical shape and which sometimes bear a heavy load. Depending on the activity (e.g., sports) and morphology, bone density varies and implants must be adapted to allow for expansion but also to bear loads. Thus, for a horse, it may be necessary to have implants with more load-bearing arms (at least 3 or 4) than for other species (where 2 support arms are sometimes sufficient). In the absence of support arms, the implant must have sufficient mechanical strength, thanks to its ability to withstand greater cement pressure than in other cases. Some embodiments comprising more than two flanges notably can prove to be more effective in the treatment of long bones of this type by distributing the expansion forces over more than two surfaces, thereby providing better stability irrespective of the type of bone.

In addition, the tibial plateau is frequently subject to crushing, and the implants or systems of the present application are useful for restoring height in any type of bone crushing or collapse, for example in the distal part of the humerus or femur. On the other hand, as taught, for example, in document EP2921142, it is possible to use expandable implants as bone anchoring implants, and such use is also possible for implants such as those of the present application. In this case, the implants will be extended at their proximal end by an elongated body to which another orthopedic implant of another type or a surgical device for fixing other elements can be attached. Nevertheless, in the case of use as a bone anchor in a vascularized structure, such as a humeral or femoral head, the size of the implant will preferably be limited in relation to the bone structure in order to preserve vascularization and promote bone healing.

Some embodiments anticipate the injection of a fluid (for example, “bone cement”, generally based on a polymer such as PMMA, for example, and well known to a person skilled in the art, such that no details concerning the cement will be provided herein). Thus, once positioned, the implant notably can be stabilised by such an injection of cement. However, because cement leakages are still a significant problem in this field, various embodiments propose containing the cement in a fluidtight casing, the post-injection volume of which can be controlled by virtue of the structure and the material of the casing, as a function of the injected pressure (and the configuration of the bone tissues, preferably assessed in ad-vance, as is the general practice in this field). Of course, fluidtightness is relative and this term is not limiting either, since the level of fluidtightness is actually adapted to the viscosity of the cement when it is injected. Some embodiments particularly allow proportional expansion of the casing by virtue of the (relative) flexibility of the sheet (10) of biocompatible metallic material. This material is generally a titanium alloy that is obtained in the form of a very thin sheet, preferably by rolling in order to yield a controlled surface condition and a controlled thickness, notably a thickness ranging between 3 and 100 microns, generally between 6 and 50 microns and preferably 10 and 30 microns.

Generally, the present invention uses at least one sheet (10) of biocompatible metal or biocompatible metal alloy, such as titanium or its alloys, particularly with nickel or others, but also nitinol or stainless steel or their alloys. Advantage is taken of recent techniques for obtaining very thin sheets of such metals, in particular with a thickness of less than 50 or even 40 μm, which makes it possible to obtain relatively flexible and elastic sheets, but above all, the plastic deformation of which can be used reversibly without reaching their tear limit, by creating folds arranged longitudinally on the implant. In particular, it is possible to provide a maximum unfolded volume that is greater than the volume required for the desired applications, so that this limit is never reached and it is possible to fold and then unfold the implant, even several times (for example, in the event of incorrect positioning of the implant), without the risk of uncontrolled tearing and leakage. Thus, thanks to this type of sheet and the configuration of their interlocking folds, it is possible to obtain expansion ratios between the folded volume and the deployed volume ranging from 2 to 20, or even 30, and it is also possible to control the shape of the implant in the deployed configuration, depending on the arrangement of the folds, in the manner of origami. Finally, although the main goal here is to prevent cement leakage, it can sometimes be useful to control the release of cement outside the implant, so that it is no longer a question of leakage but of controlled release, for example to allow adhesion to certain surrounding structures (usually bone structures). Similarly, as the injected fluid is not necessarily cement (or at least not the fluid that would come out of the implant), it may in fact be useful to administer molecules through such controlled release of this fluid. Thus, various embodiments provide for a certain porosity of the sheets (10) at least in certain portions of the implant, for example through holes of controlled microscopic size and controlled number and density. In any case, this type of sheet is capable of reversible plastic deformation for a number of times that is satisfactory for the target application, since it notably offers the possibility of retracting the casing formed by the sheet in the event of a problem (biocompatibility and resistance to tearing). Indeed, in general, controlling the metering of the cement allows the fifteen minutes of polymerisation time to be monitored, during which time it is possible to retract the casing and aspirate the cement. Furthermore, through the injection of cement and the expansion of the casing the implant fills the spaces in the damaged tissues as a function of the compression and bone-resistance forces relative to the hydraulic pressure supplied during the injection of cement. A closed structure needs to be obtained from such a sheet, which already means that the sheet needs to be closed on itself and locked in position. To this end, welding (or bonding or brazing, with these terms being non-limiting herein) can be used to join together two superposed edges or edges with interlocking turnups, in order to facilitate the welding and make it more robust. Some embodiments therefore contemplate closure by welding from the outside, which is simpler and more robust because of the superposition of layers in the vicinity of these complementary folds.

Various methods of implementation make it possible to obtain an expandable implant with very small dimensions in the collapsed configuration while ensuring a satisfactory volume in the expanded configuration. Thus, the passage required for the introduction of the implants of the present application is generally smaller than that of known implants, while the expansion is greater than that of these known implants. In fact, the folded diameter or volume is less than the deployed diameter by a factor of between 3 and 20, generally 3 to 8, preferably 4 to 7. This ratio obviously depends on the amount of cement injected, and certain embodiments take advantage of the fact that it is possible to design an implant capable of deploying more than necessary, in particular by retaining folds in the deployed configuration. Thus, the volume of the implant will be determined based on the reduced size necessary for insertion into the bone tissue and therefore with reference to the folded volume. However, different volumes are provided for the deployed configuration, since the number of folds and the length of the folds allow the deployment ratio to be increased.

The term “secured” in this case means that the two elements are secured to one another, either permanently (or quasi-permanently), but also sometimes means that a connection is made so that one element can be actuated by another. Thus, screw-fastening or collaboration between shapes for temporarily locking the elements together are covered by this non-limiting term.

The terms ring, sleeve, or tube refer to hollow structures such as bands, conduits or pipes, but in a non-limiting manner, notably assuming various shapes (on the inside as on the out-side), although a cylindrical shape is preferred. The term canal by contrast is preferably used herein to refer to a passage rather than to the element that contains it, and the term opening in this case refers to the fact that an element is open and able to be passed through, emerging into another structure or another element. In general, the terms sleeve, tube or conduit refer to longer elements than rings or bands, although their use herein likewise is non-limiting. Furthermore, the terms socket or cup also refer to hollow structures that are open at one end but are closed at the other end, such as plugs, closures, constrictions or re-strictions, and these terms are used indiscriminately without any limitation.

Certain embodiments relate to instrumentation for at least one expandable bone implant (1) for veterinary orthopedic surgery for restoring the volume and/or geometry of a bone by expanding between a collapsed configuration and an expanded configuration, said implant comprising a hollow body extending along a longitudinal axis (L) between a proximal end (11) connectable to an implantation instrument (A) for holding the implant and a distal end (12) intended to be inserted first into the bone. This proximal end can be connected to a gripping instrument (known as an implant holder) and is therefore capable of cooperating with the latter by means of physical attachment or connection means, for example known to those skilled in the art. Nevertheless, certain embodiments provide specific and advantageous attachment means to facilitate the gripping of the implant by an implant holder and, above all, the release of the implant by an L-shaped movement of the implant holder. On the other hand, by being connectable to the instrument, the implant is generally operable for expansion (here by injecting a fluid inside), as is widely known in the prior art. Indeed, many systems include expandable implants that can be actuated when mounted on an implant holder that includes an actuating means for expanding the implant (generally a conduit and/or a rod passing through the implant holder to open into a cavity in the implant and/or cooperate with an implant component that allows its expansion, the actuation generally involving a pushing and/or pulling force). The skilled person will therefore understand from reading this application that the implant can be defined without further detail on the instrument and on the actuation, since these are conventional mechanisms in the field and the system comprising the implant and the instrument is of course fully defined, but that the the implant alone is in fact also well defined in its operable nature independently of the instrument and without unnecessary detail on the actuation mechanism (sliding rod, for example), since these are perfectly standard or conventional mechanisms in the field. It is understood in the field of the present application that the term “actuable” implies pushing or pulling, and the present application thus provides sufficient explanation for the implant to be considered sufficiently clearly defined without additional reference to the instrument enabling its actuation. On the other hand, certain embodiments may relate to the instrument itself, through the originality of its elements allowing the implant to be grasped and/or actuated for expansion, and these characteristics then define the instrument independently of the implant, since they do not require any particular details about the implant other than those relating to the function performed by the instrument.

In general, it is understood that the implant will retain, even in the deployed configuration, at least a portion of folds lying flat and rolled up near the proximal and distal ends, but the dimensions and strength properties of the sheet (10) used allow the implant to be obtained and these persistent folds do not interfere with function and do not cause mechanical or physiological problems in the bone tissue. In certain embodiments, the implant comprises, in the deployed position, a middle portion between its two ends which has a generalized cylindrical shape, with possible and at least partial persistence of said folds, said middle portion extending, on the proximal end side (11), by a truncated cone portion connecting the middle portion to said sleeve and, on the side of the distal end (12), by a truncated cone portion connecting the middle portion to said socket, the truncated cone portions having a permanent persistence of at least a portion of folds lying flat and rolled up near the proximal (11) and distal (12) ends.

The terms instrumentation and instrument are generally used herein to refer to surgical intervention tools for implanting at least one expandable implant, and the set of tools is preferably referred to by the term instrumentation, while each individual tool is preferably referred to using the term instrument, generally followed by its main function or even re-placed by its function, as in the case, for example, of an implant holder, which is a known term used, for example, to refer to ancillaries used to hold implants when they are implanted.

It also should be noted that the fluid injection instrument (Ac) can be provided with means for controlling the pressure and/or the injected amount (a pressure gauge or at least gradations, for example) and for indicating the resulting volume, so as to effectively control the expansion into the bone tissues. Advantageously, means for controlling the injected air can be present in order to adapt the cement injection as a function of the air discharged in the hollow tubes or cannulas of the instruments (with the air generally easily escaping from the implant to the instrument by virtue of the clearance between the parts (rods and tubes or cannulas) of the instrument).

Finally, it is understood that the instrumentation proposed in the present application in some embodiments, using a relatively conventional implant holder (or ancillary) to hold the implant and introduce it into the bone tissues, but also that is less conventional for expanding it into the bone tissues, also offers the advantage that all the implantation and stabilisation steps can be carried out with a single instrument and in a continuous operation. Indeed, the ancillary with a hollow tube for conveying cement through the tube that retains the cement allows an instrument to be provided that allows the surgical intervention to be performed quickly and efficiently. After drilling, the implant is introduced and, without withdrawing the instrument, the casing can be inflated with the cement and then the tool can be withdrawn before, during or even after the polymerisation of the cement (for example, using a mechanism for cutting the hardened cement as the instrument rotates). The time taken to perform the surgical operation is clearly markedly reduced, but also the stability of the implant is improved, which implant is not released at any time until it has been stabilised by the injection of cement filling all the free volumes around it, unlike in some solutions of the prior art.

In general, the present invention relates to an instrument (A) for implanting expandable bone implants (1) for veterinary orthopaedic surgery, for restoring the volume and/or the geometry of a bone, by expanding between a folded configuration and a deployed configuration of the implant (1) forms a sealed container containing a fluid said implantation instrument (A) for holding the implant and a distal end (12) intended to be firstly inserted into the bone, said instrument (A) comprising a main part that can be gripped by a practitioner, an implant-holding portion (AA) comprising a gripping tube (A0) extending along the longitudinal axis (L) and having, at its distal end, retention means that complement engagement means for engaging the implant, for holding the implant by its proximal end (11). This instrumentation or instrument (A) is characterised in that it comprises an instrument (Ac) for injecting fluid, such as bone cement, into said implant (1), comprising at least one cavity adapted to receive the fluid, said injection instrument (Ac) being disposed behind the implant-holding portion (AA) along the longitudinal axis (L) and comprising at least one cannula (A0, A1, A3) for conveying the fluid to the inside of the implant (1) while it is still held by said implant-holding portion (AA). An instrument opening directly into a closed envelope of an expandable implant is not currently known in the prior art, which has only proposed the idea without successfully implementing it in a realistic manner.

In some embodiments, the instrument (A) also comprises an instrument (Ae) for expanding the implant (1) adapted to engage with mechanical means for expanding the implant (1), by virtue of an expansion rod (A3) passing through said implant-holding portion (AA) and said gripping tube (A0) in order to actuate said mechanical expansion means. In some embodiments, said expansion rod (A3) passes through said injection instrument (Ac) to the distal end of the gripping tube (A0).

In some embodiments, the retention means for retaining the instrument (A) and their complementary engagement means for engaging the implant (1) comprise a proximal sleeve of the implant, around which said retention means are attached and which comprise a stud that can be moved in an L-shaped groove in order to retain the implant by translational and rotational movement of said stud in said groove relative to the longitudinal axis.

In some embodiments, said injection instrument (Ac) comprises a piston (Pc) that can be activated on the instrument (A) in order to push the fluid stored in a chamber inside the instrument (A) through said cannula (A0, A1).

In some embodiments, said injection cannula (A0, A1) is formed by the gripping tube (A0) emerging onto a proximal sleeve of the implant (1). In other embodiments, said injection cannula (A0, A1) is formed by an injection tube (A1) adapted to be inserted inside the gripping tube (A0) and emerging into an opening in a proximal sleeve of the implant (1) retained by the gripping tube (A0), directly into a cavity of the implant (1) or onto a shaft (3) of the implant (1) comprising a conduit (31) and at least one opening (32) for distributing said fluid within the implant. In yet other embodiments, said injection cannula (A0, A1) is formed by said expansion rod (A3), which is hollow and is adapted to be inserted inside the gripping tube (A0) and emerges into an opening in a proximal sleeve of the implant (1) retained by the gripping tube (A0), directly into a cavity of the implant (1) or onto a shaft (3) of the implant (1) comprising a conduit (31) and at least one opening (32) for distributing said fluid within the implant.

In some embodiments, the implant-holding portion (AA) comprises a mounting secured to the gripping tube (A0) directly in the main instrument (A). In other embodiments, the implant-holding portion (AA) comprises a detachable tip having a housing adapted to be mounted on a protrusion of the instrument (A) through which the fluid is conveyed to the detachable tip supporting the gripping tube (A0).

In some embodiments, said detachable tip forming the implant holder (AA) is retained on the instrument by virtue of a stud engaging with an L-shaped groove for locking in a translational and rotational movement relative to the longitudinal axis (L). In some of these embodiments, the direction of rotation for locking said implant holder (AA) on the instrument (A) is opposite the direction of rotation for locking the implant (1) on the implant holder. In some of these embodiments, the implant holder (AA) comprises a translation latch detachably disposed in said groove in order to prevent any translation movement of said stud, until fluid is injected and/or actuation of the expansion causes said implant (1) to shorten in length. Furthermore, in some of these embodiments, the implant holder (AA) comprises a rotation latch detachably disposed in said groove in order to prevent any rotation movement of said stud, until the uncoupling of the implant holder (AA) and the instrument (A) is required.

In some embodiments, locking means are provided in some instances, to prevent the implant from folding on itself. The very narrow diameters of the implants and their central shafts are not easily compatible with screw threads for expanding through a screwing action on the implant, although performing a screwing action on the instrument actuating the expansion is advantageous, notably when the expansion involves bringing the support arms closer to one another. Thus, as is known from the prior art, a split ring can be used, for example, that is housed in a circular recess of the implant and engages with teeth on the push-or pull-shaft, which teeth are oriented in such a way as to allow this shaft to move in only one direction. Thus, the shaft can be actuated for expanding the flanges by rotating past successive teeth, thereby allowing the implant to be locked in the deployed configuration.

However, unlike some implants of the prior art, in which the pull-shaft providing the moving-together action necessarily has to remain in place, the implants of the present application are deployed without any moving-together of the support arms, which advantageously allows these arms to be locked with a screw lock so that such teeth that make the task difficult and offer reduced reliability are not needed. Thus, it is possible, for example, for a threaded sleeve to be used that is configured to be arranged in the hollow tube of the im-plantation instrument (A) holding the implant (and surrounding any fluid injection conduit present inside). Such a sleeve then has a screw thread intended to engage with a tapped thread of the proximal end of the implant and has actuating means for screwing or un-screwing.

In some embodiments, the instrument (A) for implanting and for injecting (Ac) cement comprises means for controlling the pressure and/or the aspiration of the cement so that the implant can be folded to the folded configuration if necessary.

In some embodiments, the implantation instrument (A) is distinct from but complements the injection instrument (Ac), the cement-injection canal of which passes through a canal inside the rod of the implantation instrument (A) holding the proximal end of the implant (1) via its distal end.

The illustrative and non-limiting figures of the present application will now be described in detail in order to better explain the various embodiments and to provide examples of structural elements that can be used in the foregoing context. Therefore, the following description must not be considered to be limiting, since the various illustrated elements or components are merely examples and the figures can combine elements or components that are not necessarily dependent on one another.

FIG. 1A shows a profile view of instrumentation for implanting an expandable orthopaedic implant according to some embodiments, comprising an implant holder, a cement injection instrument and the implant supported at the end of the implant holder, and FIG. 1B shows a perspective view of the instrumentation of FIG. 1A with the cement injection instrument removed from the implant holder.

FIG. 1A shows a profile view of instrumentation for implanting a deployable implant according to some embodiments, with an implant (1) mounted on an implant holder (1) assembled with a cement injection instrument (Ac) towards the inside of the implant. This instrumentation (A, Ac) comprises a loading port (Oc) for loading cement into the body of the instrument and an air bleed valve (PE) for expelling air when loading cement through the loading port. This valve, which has a truncated frustoconical portion that is particularly visible in FIG. 8B, can be screwed in to close the compartment into which the cement has been loaded once the air has been expelled. The cement injection instrument (Ac), as shown in FIG. 1B, preferably comprises a handle for screwing in a piston with a threaded part and a smooth part provided with seals, so that the piston secured to the handle can be advanced and the cement can be pushed through the instrumentation. The implant holder comprises a hollow body adapted to receive the piston and a tapped thread for engaging with the thread. However, the injection of cement can be designed for the use of pushing means other than screwing, such as pressure means, for example, notably hydraulic pressure. The key point is to be able to push the cement through a cannula A0 into the implant to allow the volume of a casing (10) of the implant (1) to increase in order to expand and fill the casing, for example, as shown in FIG. 1A, by controlling the injected volume using the injection instrument (via the screw handle in this example and preferably with gradations allowing the injected volume to be quantified).

FIG. 2A shows a partial cross-sectional or transparent view of instrumentation for implanting an expandable implant, FIG. 2B shows a perspective view with a partial cross-section of the instrumentation of FIG. 2A, and FIG. 2C shows a perspective view of the end of the implant holder with an implant mounted thereon according to some embodiments. FIG. 2A shows the cement injection instrument pushed to the end of its stroke, once the casing is fully deployed, and FIG. 2B shows the seals in the vicinity of the loading port (OC), into which the cement can be introduced by expelling any air via the bleed valve (PE). Once the forward movement of the piston of the injection instrument has pushed the cement to the distal end of the instrument, the cement is directed towards a cannula (A0) on the implant holder, which is used to hold the implant. This cannula (A0) of the implant holder also freely translationally accommodates a hollow cannula (A1), through which the cement can pass in order to be injected into the implant. This cannula (A1) actually has a distal end that engages with a hollow cup closing the implant at its distal end (12). The distal end of the injection cannula (A1) comprises either a plug (A12, FIGS. 2C and 8B) closing the cannula or an open end for injecting cement. The plug (A12) prevents, when the injection cannula (A1) is withdrawn, as is partially shown in FIG. 2C, once the cement is injected, when the holes (A2) in the cannula are sealed inside the cannula (A0) of the implant holder, the implant holder from being permeated with cement and this cement is instead confined within the implant and polymerises without reaching the implant holder. If the end is open without such a plug (A12), the cannula then needs to be designed to be breakable and optionally provided with means for cutting or breaking the polymerised cement it contains when the implant is released into the implantation site. FIG. 8B shows a cross-sectional view of this end of the instrumentation with the plug (A12) sealing the distal end according to some embodiments. In other embodiments, the distal end of the cannula (A1) is open so that the cement can be injected through this end, and the holes (A2) then can be omitted, but it is still preferable for such holes (A2) to be used to allow the cement to exit laterally inside the implant, around the circumference of the cannula (A1) and over a significant portion of the size of the implant.

FIG. 3A shows a perspective view of instrumentation for expanding an expandable implant according to some embodiments inserted inside a cement injection instrument, according to some embodiments, and FIG. 3B shows the expansion instrument alone. FIG. 3A shows other embodiments of instrumentation in which the implant holder and the injection instrument are supplemented by an implant expansion instrument (Ae), according to other embodiments, which can be deployed by a mechanical expansion mechanism. This expansion instrument (Ae) comprises an expansion wheel (ME) for controlling the expansion of the implant (by its position when screwing in the expansion instrument or by adjusting the position of the wheel on the instrument). This wheel actually allows the forward movement to be adjusted of a rod or cannula (A3) disposed in the centre of the other cannulas (A0, A1) and extending into the implant in order to push or pull on mechanical elements of the implant and allow the deployment thereof. FIG. 3B shows this expansion instrument inserted through the injection instrument with its expansion wheel (ME), and FIG. 3C shows this same expansion instrument alone, thus better illustrating the role of the wheel in adjusting the depth to which it can be pushed through the rest of the instrumentation.

FIG. 9A shows a cross-sectional view of part of instrumentation for implanting an expandable implant of the type shown in FIG. 3A, and FIG. 9B shows a cross-sectional view of the implant held by such an instrument of FIG. 9A, according to some embodiments. FIG. 8A shows a cross-sectional view of part of instrumentation for implanting an expandable bone implant of the type shown in FIG. 4A, and FIG. 8B shows a cross-sectional view of the implant supported by an instrument of FIG. 8A, according to some embodiments. FIGS. 9A and 9B show cross-sectional views of various proximal parts of this expansion instrument and the path of its components through the other two instruments, respectively the cement injection and implant holding instruments. Thus, it can be seen that such an expansion instrument passes through the two instruments at the centre thereof in order to reach the implant (1) supported at the distal end. FIG. 9B shows a cross-sectional view of this instrument with a non-adjustable expansion wheel, but its screw thread nevertheless can be identified by any markings or by a known number of turns corresponding to a specific distance, with it being understood that this is generally a few millimetres or centimetres. FIG. 8B also shows details of the cement injection holes (A2), shown in FIG. 2C, with a cross-sectional view of the distal end of the instrumentation and the holes A2 present inside an implant (1) in a folded configuration.

FIG. 4A shows a perspective view of instrumentation for implanting an expandable implant comprising an implant holder mounted on a gripping instrument (A) according to some embodiments, and a cement injection instrument (Ac) passing through the assembly, FIG. 4B shows details of the circular part indicated in FIG. 4A, and FIGS. 4C and 4D show the same details after the removal of the latches on the implant holder. In particular, FIG. 4A shows a perspective view of a gripping instrument (A) comprising a grippable handle and on which an implant holder (AA) and cement injection device are detachably mounted with the implant (1) held at the distal end. This figure particularly shows the presence of a loading latch (LC) for closing the cement loading port (Oc), but also the air bleed valve (PE). FIG. 4B shows an exploded view of the zone circled in FIG. 4A, notably showing the bleed valve and the detachable implant holder (AA) mounted on the gripping instrument (A). The implant holder section features a translation stop (BT) and a translation latch (LT), as shown in FIG. 4C. Indeed, the translation stop (BT) of the instrument is locked by a detachable latch (LT) that prevents any translation movement of the implant holder (AA) relative to the gripping instrument (A). FIG. 4D shows a detachable rotation latch (LR) that also reversibly prevents the implant holder (A) from rotating on the gripping instrument, so that any translational and rotational movement of the implant holder releases the implant held by the implant holder via an L-shaped mechanism requiring a rotation and then a translation movement. FIG. 8A shows a cross-sectional view of this same end with this translation stop (BT) and the translation latch (LT), and FIG. 5B shows the engagement of the implant holder (AA) with the implant via this L-shaped retention mechanism.

FIG. 5A shows, on the left-hand side, a top view of instrumentation for implanting a bone anchor implant and, on the right-hand side, the implant holder uncoupled from the gripping instrument, and FIG. 5B shows details of the circular zone on the right-hand side of FIG. 5A, with the engagement between the implant holder and the implant, according to some embodiments. In particular, FIG. 5A shows a top view of a gripping instrument (A) with the cement injection instrument (Ac) on the left-hand side, while the right-hand side only shows the implant holder part (AA), which is adapted to be mounted at the end of the gripping instrument and to be held in place by virtue of the translation (LT) and rotation (LR) latches, in some embodiments. FIG. 5B shows an exploded view of the enclosed circle on the right-hand side of FIG. 5A showing the implant holder (AA) and shows that the cannula (A0) of the implant holder (AA) comprises an internal conduit provided with projections adapted to engage with an L-shaped groove formed on the proximal end (AA) of the implant (1). Thus, it is understood that FIGS. 4A to 4D and FIG. 5B il-lustrate the role of the translation latch (LT) and of the rotation latch (LR) in holding the implant during implantation and releasing the implant once implantation is complete, notably with the injection of cement, which is generally completed by removing the injection cannula A1. Indeed, the proximal end of the implant comprises an L-shaped groove that allows the implant to be gripped by a tool comprising a gripping means that can be activated by a quarter-turn movement matching the L-shape, thereby facilitating assembly with the implant holder and, especially, the release of the implant on completion of the operation. The term quarter turn can be used herein to refer to a conventional mechanism, but it is obvious that such a means does not necessarily require a 90°rotation and that it can be more or less, although a significant rotation is preferable before the implant can be released.

FIGS. 6A, 6B and 6C show perspective views of vertebrae into which various embodiments of expandable bone implants are inserted by virtue of instrumentation according to various embodiments. In FIG. 6A, the implant only comprises a deployable casing and does not require a mechanical expansion instrument (Ae), and the instrumentation therefore only comprises the cannula (A0) of the implant holder (AA) and the cement injection cannula (A1), whereas in FIGS. 6B and 6C, the implants comprise mechanical expansion mechanisms and require a cannula (A3) for actuating an expansion instrument (Ae).

FIGS. 7A, 7B and 7C show profile views of vertebrae that have respectively suffered anterior, median and posterior vertebral compression fractures (VCF). The invention allows these kinds of vertebral fractures to be treated by arranging the deployable implant in the correct position in the plane of the implantation site, using antero-posterior and/or medio-lateral positioning and by adjusting the depth of insertion and/or the angle of insertion of the implant, according to the type of surgical approach that is used (for example, lateral, anterior, dorsal, transforaminal, transpedicular, etc.) In some embodiments, the implant can comprise a second sheet (10b) surrounding the first sheet, made from the same material or another material, in order to form a double casing, for example as shown in FIG. 2D. Such a double casing can offer numerous assorted advantages, notably of providing thermal insulation to protect the tissues from the heat from polymerisation (by virtue of, for example, a fluid that limits heat transmission) or simply of providing additional safety in order to avoid any cement leakage if one of the sheets tears. In this case, at least one of the ends of the implant, in particular the proximal end (11), may comprise an additional ring or cap concentric with the first ring or cap or, for example as shown in FIG. 2D, a double-channel ring or a double ring, to secure this second sheet (10b) while maintaining a space between it and the first sheet (10), but it is possible to join these two sheets (10, 10b) together at the ends.s. In the case of two more widely spaced sheets, it is possible to provide an injection inlet between the two sheets (10, 10b) for a fluid that is different from or identical to the first fluid, for example via a double ring or a single ring with two conduits. Such a double ring may, for example, comprise spacers between a first ring and a second ring (11b) which is concentric with the first to form between them an annular section conduit allowing the injection of this second fluid. (such as, for example, a lubricating fluid that improves the ability of one sheet to slide relative to the other, thereby facilitating deployment). Of course, other configurations are possible as long as they include a duct opening into the envelope formed by the first sheet and a duct opening into the space between the two sheets. During manufacturing, these two sheets then can be folded and rolled at the same time or successively, but their welds (or crush bonds), between themselves and/or to the ring and/or to the base, shall be made successively to ensure that the space between them is maintained. These double-leaf configuration methods allow the injection site to be preformed (by compressing spongy bone tissues) but can also allow, for example, said fluid to be injected in two stages for better adjustment of the shape, of the resulting temperature in the tissues and/or of the rate of polymerisation of the fluid (for example, by adjusting the mix of the compounds of the cement). On the other hand, since it is possible to use a second fluid other than cement inside, it is possible to use the compartment between the two sheets as a cooling circuit by circulating a fluid during the polymerization of the cement, so as to protect the tissue from the heat generated during said polymerization. Such a double-sheet implant (1) therefore requires a double cannula comprising two concentric or parallel conduits, each emerging into one of the spaces provided as a person skilled in the art will understand from the FIG. 2D without the need for further explanation.

The instrumentation of the present application can be used for the implantation of various types of implants. For example, FIG. 10A shows a perspective view of an expandable implant that can be used with instrumentation according to certain embodiments, such as those shown in FIGS. 9A and 9B, comprising an expansion instrument (Ae), in particular with an expansion rod (A3). In FIG. 10A, the implant is in a collapsed configuration, and FIG. 10B shows a perspective view of the implant in FIG. 10A in an expanded configuration. In FIG. 10A, it can be seen that the expansion ring has an L-shaped groove allowing the implant to be gripped by a tool comprising a gripping means that can be actuated by a “quarter-turn” movement complementary to the L-shape, facilitating assembly with the implant holder and, above all, release of the implant at the end of the operation. The term quarter turn can be used here to refer to a conventional mechanism, but it is clear that such a means does not necessarily require a 90° rotation and that it may be more or less, although it is preferable to have a significant rotation before the implant can be released. In FIG. 10A, but more clearly in FIG. 10C, which shows a detail of the implant, it can be seen that the proximal end (11) has a conduit (31) for injecting cement into the implant, and in FIGS. 10A, 10B, and 10C, it can also be seen that the central shaft (3) has holes (32) designed to allow the cement injected via the proximal end to reach the interior of the shaft (3), which is equipped with a central conduit (31) that opens onto the holes (32), in order to facilitate the distribution of the cement inside the implant and in particular the space created by the deployment of the implant through the separation of the plates.

Other examples of expandable implants that can be used with instrumentation of the type illustrated in FIGS. 9A and 9B of this application are shown in FIGS. 11A and 11B for certain embodiments where the implant comprises a sealed envelope welded to the expansion arms of the implant, but also in FIG. 11C for other embodiments where the implant is integrated into a separate envelope, for example as in the embodiments of FIGS. 10A, 10B, and 10C. FIG. 11A shows a perspective view of an expandable implant in its folded configuration, according to certain embodiments, and FIG. 11B shows a perspective view of the same implant in its deployed configuration. In FIG. 11B, it can be seen that the sheet (10) is welded to the arms (131-141) between the plates, which are therefore free to move apart, and the sheet will conform to the shape of the implant as it expands. In these examples, the expandable bone implant (1) for veterinary orthopedic surgery for restoring the volume and/or geometry of a bone, by expanding between a folded configuration and a deployed configuration, said implant (1) extending along a longitudinal axis (L) between a proximal end (11) connectable to an implantation instrument (A) for holding the implant and a distal end (12) intended to be inserted first into the bone, at least two faces of the implant, for example upper and lower, each comprising a plate (13, 14) for contact with bone tissue, each of the plates comprising a central portion (130, 140) connected, via at least one hinge, to at least one pair of support arms (131, 141) each oriented in opposite directions within each pair, one arm of each pair being hinged to the distal end (11) while the other arm is hinged to the proximal end (12), the implant (1) comprising a central shaft (3) or a housing adapted to receive such a shaft extending through a sliding sleeve at the proximal end (11) to a traction ring or sleeve at the distal end (12) where said shaft is configured to transmit traction, when actuated by said instrument (A), on the distal end (12) to allow it to be brought closer to the proximal end (11), causing the support arms (131, 141) to pivot, causing the plates (13, 14) to move away relative to each other and, consequently, the expansion of the implant between the folded configuration and the deployed configuration.

In the examples of FIGS. 11A and 11B, the implant (1) is configured such that:
at least two other faces of the implant, between those comprising the plates, are covered with at least one sheet (10) per face, made of a biocompatible metal alloy, and sealed to the central portions (130, 140) under the plates, to the lateral faces of the arms (131, 141) and to lateral faces of the proximal end (11) and the distal end (12),
said sheet (10) is plastically deformable to allow expansion of the implant and has, at least in the folded configuration, a plurality of anti-form folds (101), known as convex folds, and synform folds (102), known as concave folds, said folds lying on top of each other in the folded configuration, the total surface area of said sheet being greater than or equal to the lateral surface area of the implant in the deployed configuration so as to form a sealed compartment adapted to receive a fluid inside the cavity obtained by the expansion of the implant.

In the example of FIG. 11C, the implant (1) is configured so that it comprises an envelope enclosing said implant from the proximal end (11) to the distal end (12) and so that:
said envelope is formed by a sheet (10) of a biocompatible metal alloy, sealed onto itself in a watertight manner;
said sheet (10) has, at least in the folded configuration, a plurality of pairs of folds, each pair comprising an antiform fold (101), known as convex, and a synform fold (102), known as concave, said folds being laid on top of each other in the folded configuration so that the surfaces between each of said convex and concave folds are wrapped around the longitudinal axis (L);
said proximal end (11) extends into a sealing sleeve that is sealed to the folds lying and wrapped around said sheet (10) around the entire periphery of the proximal end (11);
said distal end (12) extends into a sleeve sealed to the flattened and rolled folds of said sheet (10) around the entire periphery of the distal end (12) of said implant (1);
said sheet (10) is plastically deformable to allow the implant to expand from the folded configuration to the deployed configuration, forming a sealed envelope enclosing the implant and preventing leaks when fluid is injected inside the implant (1) and the envelope.

It is therefore understood from the present application that the proposed instrumentation allows expandable implants to be implanted in bone tissue and a fluid to be injected directly into the implants in a single operation without releasing the implant and/or the instrument between expansion and injection, thereby avoiding any risk of misplacement during removal (except for the variants in FIGS. 8A and 8B, where it is necessary to remove the expansion instrument before adding the injection instrument). In fact, when it comes to an implant such as those shown in FIGS. 2A, 2B, 2C, and even 2D, the instrumentation allows the implant to be deployed (expanded) automatically by injecting fluid, unlike the prior art, where a flexible balloon is generally used first and then removed before injecting polymerizable cement. This type of implant that can be used with the instrumentation of the present application can be defined as an expandable bone implant (1) for veterinary orthopedic surgery for restoring the volume and/or geometry of a bone, by expansion between a folded configuration and an expanded configuration, said implant comprising a hollow body extending along a longitudinal axis (L) between a proximal end (11) connectable to an implantation instrument (A) for holding the implant (1) and a distal end (12) intended to be inserted first into the bone. This type of implant (1) is generally configured such that:
the wall of said hollow body is formed by a sheet (10) of a biocompatible metal alloy, sealed onto itself between said proximal (11) and distal (12) ends;
said sheet (10) has, at least in the folded configuration, a plurality of pairs of folds, each pair comprising an antiform fold (101), known as convex, and a synform fold (102), known as concave, said folds lying on top of each other in the folded configuration so that the surfaces between each of said convex and concave folds are wrapped around the longitudinal axis (L),
said proximal end (11) comprises a ring sealed to the folds lying and wrapped around said sheet (10) over the entire periphery of the proximal end (11), the opening through the ring providing an entrance to the interior of the hollow body of the implant (1),
said distal end (12) comprises a base closing the distal end (12) and sealed to the flat and rolled folds of said sheet (10) around the entire periphery of the distal end (12) of said hollow body
said sheet (10) being plastically deformable to allow the implant to expand from the folded configuration to the deployed configuration when a fluid is injected into the implant (1).

Similarly, in the case of an implant such as those shown in FIGS. 10A, 10B, 11B, or 11C, it is first necessary to expand the implant using an expansion rod (A3) and then inject the cement, but the instrumentation of the present application, and in particular that of FIGS. 9A and 9B, allows the cement to be injected without having to remove any element and therefore ensures unparalleled stability and reliability during the surgical procedure. In all these examples, with the mechanical expansion of the implant, the injection of fluid stabilizes the implant and even locks it in the deployed configuration when the fluid is a polymerizable cement such as those known in the prior art. It is therefore understood that the present application also relates to a system comprising the instrumentation described herein, in combination with at least one implant as described herein, but that it is not essential to limit the scope of the invention to a system insofar as the instrumentation disclosed in the present application is suitable for a large number of implants, the main characteristics of which necessary for the implementation of the present invention are perfectly accessible to the skilled person, such as the actuation of mechanical means for expanding the implant. In the case of a simple injection of fluid into a balloon, those skilled in the art will understand that the instrument is specific in that it is adapted to retain the implant itself for a single-step operation instead of retaining or not retaining the implant at the same time as a balloon is inflated. With regard to implants, it should be noted that, in general, the number of pairs of folds is between 3 and 16, usually 4 to 12, preferably around 8. However, 3 folds may sometimes be sufficient, but the greater the number of folds, the less the material will deform, the less risk there will be of tearing, and the easier it will be to deploy. Thus, it is possible to plan for up to 20 folds, even for medium-sized animals, and when it comes to large animals such as horses, it is possible to allow for 25 to 30 folds, or even more.

The present application describes various technical features and advantages with reference to the figures and/or to various embodiments. A person skilled in the art will appreciate that the technical features of a given embodiment actually can be combined with features of another embodiment unless otherwise explicitly stated or unless it is obvious that these features are incompatible or that combining them would not provide a solution to at least one of the technical problems cited in the present application. In addition, the technical features described in a given embodiment can be taken in isolation from the other features of this embodiment unless otherwise explicitly stated.

### DETAILED LIST OF REFERENCES IN THE FIGURES

1 implant
10 sheet
10b second sheet 11b second ring
11 proximal end
101 antiform fold
102 synform fold
110 proximal weld
12 distal end
120 distal weld (fluidtight connection)
121 compression fixing (for example, split ring)
3 central shaft
31 central-shaft conduit
13 first flange
14 second flange
15 third flange
20 expansion ring (or sleeve) 131 first-flange support arm
141 second-flange support arm
151 third-flange support arm
132 first-flange expansion arm
142 second-flange expansion arm
152 third-flange expansion arm
130 first-flange central support arm
140 second-flange central support arm
150 third-flange central support arm
A implantation instrument
AA implant holder
Ae expansion instrument
Ac fluid-injection instrument
A1 hollow gripping tube
A0 (or Ao) grasping tube (or cannula)
A1 injection tube (or cannula)
A2 injection holes
A3 expansion rod
A12 distal plug

1. Instrument (A) for implanting expandable bone implants (1) for veterinary orthopaedic surgery, for restoring the volume and/or the geometry of a bone, by expanding between a folded configuration and a deployed configuration of the implant (1) extending along a longitudinal axis (L) between a proximal end (11) adapted to engage with said implantation instrument (A) for holding the implant and a distal end (12) intended to be firstly inserted into the bone, said instrument (A) comprising a main part that can be gripped by a practitioner, an implant-holding portion (AA) comprising a gripping tube (A0) extending along the longitudinal axis (L) and having, at its distal end, retention means that complement engagement means for engaging the implant, for holding the implant by its proximal end (11), wherein said instrument (A) comprises an instrument (Ac) for injecting fluid, such as bone cement, into said implant (1), comprising at least one cavity adapted to receive the fluid, said injection instrument (Ac) being disposed behind the implant-holding portion (AA) along the longitudinal axis (L) and comprising at least one cannula (A0, A1, A3) for conveying the fluid to the inside of the implant (1) while it is still held by said implant-holding portion (AA).

2. The instrument (A) according to claim 1, further comprising an instrument (Ae) for expanding the implant (1) adapted to engage with mechanical means for expanding the implant (1), by virtue of an expansion rod (A3) passing through said implant-holding portion (AA) and said gripping tube (A0) in order to actuate said mechanical expansion means.

3. The instrument (A) according to claim 2, wherein said expansion rod (A3) passes through said injection instrument (Ac) to the distal end of the gripping tube (A0).

4. The instrument (A) according to claim 1, wherein the retention means for retaining the instrument (A) and their complementary engagement means for engaging the implant (1) comprise a proximal sleeve of the implant, around which said retention means are attached and which comprise a stud that can be moved in an L-shaped groove in order to retain the implant by translational and rotational movement of said stud in said groove relative to the longitudinal axis.

5. The instrument (A) according to claim 1, wherein said injection instrument (Ac) comprises a piston (Pc) that can be activated on the instrument (A) in order to push the fluid stored in a chamber inside the instrument (A) through said cannula (A0, A1).

6. The instrument (A) according to claim 1, wherein said injection cannula (A0, A1) is formed by the gripping tube (A0) emerging onto a proximal sleeve of the implant (1).

7. The instrument (A) according to claim 1, wherein said injection cannula (A0, A1) is formed by an injection tube (A1) adapted to be inserted inside the gripping tube (A0) and emerging into an opening in a proximal sleeve of the implant (1) retained by the gripping tube (A0), directly into a cavity of the implant (1) or onto a shaft (3) of the implant (1) comprising a conduit (31) and at least one opening (32) for distributing said fluid within the implant.

8. The instrument (A) according to claim 1, wherein said injection cannula (A0, A1) is formed by said expansion rod (A3), which is hollow and is adapted to be inserted inside the gripping tube (A0) and emerges into an opening in a proximal sleeve of the implant (1) retained by the gripping tube (A0), directly into a cavity of the implant (1) or onto a shaft (3) of the implant (1) comprising a conduit (31) and at least one opening (32) for distributing said fluid within the implant.

9. The instrument (A) according to claim 1, wherein the implant-holding portion (AA) comprises a mounting secured to the gripping tube (A0) directly in the main instrument (A).

10. The instrument (A) according to claim 1, wherein the implant-holding portion (AA) comprises a detachable tip having a housing adapted to be mounted on a protrusion of the instrument (A) through which the fluid is conveyed to the detachable tip supporting the gripping tube (A0).

11. The instrument (A) according to claim 10, wherein said detachable tip forming the implant holder (AA) is retained on the instrument by virtue of a stud engaging with an L-shaped groove for locking in a translational and rotational movement relative to the longitudinal axis (L).

12. The instrument (A) according to claim 11, wherein the direction of rotation for locking said implant holder (AA) on the instrument (A) is opposite the direction of rotation for locking the implant (1) on the implant holder.

13. The instrument (A) according to claim 10, wherein the implant holder (AA) comprises a translation latch detachably disposed in said groove in order to prevent any translation movement of said stud, until fluid is injected and/or actuation of the expansion causes said implant (1) to shorten in length.

14. The instrument (A) according to claim 10, wherein the implant holder (AA) comprises a rotation latch detachably disposed in said groove in order to prevent any rotation movement of said stud, until the uncoupling of the implant holder (AA) and the instrument (A) is required.

15. A veterinary orthopedic surgery system comprising at least one expandable bone implant (1) for restoring the volume and/or geometry of a bone, by expansion between a collapsed configuration and an expanded configuration of the implant (1), and at least one instrument (A) for implanting said implant, characterized in that said instrument of said system is an instrument according to claim 1.
